(19) United States
(12) Patent Application Publication
(10) Pub. No.: US 2009/0057549 A1
(43) Pub. Date: Mar. 5, 2009

(54) Method of Excising Sugar Chain from Glycoprotein, Method of Mass Spectrometry of Sugar Chain, and Method of Mass Spectrometry of Glycoprotein

(75) Inventors: Satoshi Kimura, Kyoto (JP); Akihiko Kameyama, Ibaraki (JP); Hisashi Narimatsu, Ibaraki (JP)

Correspondence Address: Cheng Law Group, PLLC, 1100 17th Street, N.W., Suite 503, Washington, DC, 20036, US

(73) Assignees: Shimadzu Corporation, Kyoto-shi, Kyoto (JP); National Institute of Advanced Industrial Science and Technology, Tokyo (JP)

(21) Appl. No.: 11/918,405

(22) PCT Filed: Apr. 7, 2006
(86) PCT No.: PCT/JP2006/307872

§ 371 Date: Oct. 12, 2007

(30) Foreign Application Priority Data
Apr. 13, 2005 (JP) 2005-115646

(51) Int. Cl. B01D 59/44; C12P 19/00

(52) U.S. Cl. 250/282; 435/72

(57) ABSTRACT

The present invention provides a method of enzymatic reaction on a membrane by a sugar chain releasing enzyme, for an MALDI-TOF MS analysis of a glycoprotein that is solid-phased on a membrane is conducted directly on the membrane; a method of mass spectrometry of a sugar chain in which a sugar chain, for the MALDI-TOF MS analysis of a sugar chain excised from a glycoprotein that is solid-phased on a membrane is conducted directly on the membrane; and a method of mass spectrometry of a glycoprotein, for the MALDI-TOF MS analysis of a glycoprotein of a glycoprotein that is solid-phased on a membrane is conducted directly on the membrane. A method of excising a sugar chain to obtain excised sugar chains by excising the sugar chains by dispensing a sugar chain releasing enzyme solution on a glycoprotein that is solid-phased on a carrier, wherein the sugar chain releasing enzyme solution is a solution containing a sugar chain releasing enzyme in a reaction buffer solution containing a buffering agent consisting essentially of a volatile component. A method of mass spectrometry of a sugar chain and a method of mass spectrometry of a glycoprotein using the method of excising the sugar chain.

### TECHNICAL FIELD

The present invention relates to a method of analyzing the structure of a glycoprotein. In particular, the present invention relates to a method of analyzing the structure of a glycoprotein that is solid-phased on a carrier by mass spectrometry.

### BACKGROUND ART

More than half of biological proteins are modified with sugar chains. Therefore, in a protein structure analysis considering mutation in function or disease, it is necessary to analyze not only the protein itself but also a sugar chain modifying the protein. In a conventional analysis method of a glycoprotein, a structure analysis is conducted that is based on separation of a glycoprotein by 2D-PAGE, excision of a spot of a glycoprotein, peptide digestion or sugar chain excision of a glycoprotein in a tube, separation and purification of peptide digests and excised sugar chains on respective columns and so on, and an MS analysis.

On the other hand, International Patent Publication No. 98/47006, pamphlet and Molecular & Cellular Proteomics, Vol. 1, 2002, p. 490-499, have recently reported a series of analytical processes based on a microdispensing technology which involves microdispensing of trypsin by means of a microdispenser using an inkjet technique such as a piezo element to a protein which is transferred and fixed on a PVDF membrane after 2D-PAGE, an enzymatic reaction on the membrane, and dispensing of a matrix, and a peptide analysis by an MALDI-TOF-MS analysis conducted directly on the membrane.

As an application example of the above microdispensing technology, a structure analysis of a glycoprotein as will be described later is conducted in Molecular & Cellular Proteomics, Vol. 1, 2002, p. 490-499. Specifically, also for a glycoprotein that is transferred and fixed on the PVDF membrane, development by electrophoresis, an enzymatic reaction by conducting printing to a protein spot of peptide-N-glycosidase F (PNGase F) available from Roche, manual extraction of N-glycan in the spot with 1 microlitter of pure water, and a sugar chain analysis by LC-ESI MS are conducted in similar manners. On the other hand, for the protein spot from which a sugar chain is extracted, washing with pure water (for removing residual PNGase F), an enzymatic digestion reaction on the membrane by dispensing trypsin on the same spot, dispensing of a matrix and a direct peptide analysis are conducted. In this manner, the structure analysis of a glycoprotein is achieved.

Patent document 1: International Patent Publication No. 98/47006, pamphlet

Non-patent document 1: Molecular & Cellular Proteomics, Vol. 1, 2002, p. 490-499

### DISCLOSURE OF THE INVENTION

### Object of the Invention

In the above-described protein analysis method based on the microdispensing technology, the series of operations are conducted on a membrane, and effectiveness of a chemical printer method is reported.

The glycoprotein analysis method in which the above microdispensing technology is applied, however, involves complicated works such as extraction and purification of digests as N-glycan existing in the PVDF membrane is manually extracted with water and the extracts are measured by LC-ESI MS in the sugar chain analysis. Therefore, advantages in throughput by using a chemical printer are not satisfactorily exerted.

Further, in the above method, in the PNGase F treatment, printing is executed with 500 nanolitters of a 5 units/μL concentration enzymatic solution buffered with 100 mM of a sodium phosphate buffer solution, 25 mM of EDTA, pH 7.2. Because EDTA which is contained in a significant amount in the enzymatic reaction solution provides a strong peak that overlaps with a sugar chain in mass spectrometry, and inhibits crystallization of a matrix, it is difficult to directly conduct an MALDI-TOF-MS analysis on a membrane. Therefore, an MS analysis is achieved by employing an LC-ESI MS analysis method and LC separating EDTA from a sugar chain on a column.

Any of commercially available PNGase F (purchasable from Roche, TaKaRa and so on) contains a similar buffer solution as described above. Therefore, it is necessary to find a condition for an enzymatic reaction on a membrane, that will not inhibit the enzymatic reaction of PNGase F and will not influence on MALDI-TOF MS.

In view of the above, it is an object of the present invention to provide a method of enzymatic reaction on a membrane by a sugar chain releasing enzyme, for an MALDI-TOF MS analysis of a glycoprotein that is solid-phased on a membrane is conducted directly on the membrane; a method of mass spectrometry of a sugar chain in which a sugar chain, for the MALDI-TOF MS analysis of a sugar chain excised from a glycoprotein that is solid-phased on a membrane is conducted directly on the membrane; and a method of mass spectrometry of a glycoprotein, for the MALDI-TOF MS analysis of a glycoprotein of a glycoprotein that is solid-phased on a membrane is conducted directly on the membrane.

### SUMMARY OF THE INVENTION

Inventors of the present application found that the object of the present invention is achieved by using a reaction buffer solution which consists substantially of a volatile component, and accomplished the present invention.

The present invention includes the following inventions.

<1> A method of excising a sugar chain to obtain the excised sugar chain by excising the sugar chain by dispensing a sugar chain releasing enzyme solution on a glycoprotein that is solid-phased on a carrier,

wherein the sugar chain releasing enzyme solution is a solution containing a sugar chain releasing enzyme in a reaction buffer solution containing a buffering agent consisting essentially of a volatile component.

<2> The method of excising a sugar chain according to <1>, wherein the sugar chain releasing enzyme is N-glycanase or glycopeptidase A.
<3> The method of excising according to <1> or <2>, wherein the reaction buffer solution is selected from an (NH₄)HCO₃ buffer solution, a CH₃CO₂NH₄ buffer solution, an (NH₄)₂CO₃ buffer solution, and a N(CH₃CH₂)₃ buffer solution.
<4> The method of excising a sugar chain according to any one of <1> to <3>, wherein the solid-phased glycoprotein is treated with endoglycosidase or exoglycosidase other than the sugar chain releasing enzyme.
<5> A method of mass spectrometry of a sugar chain which detects the excised sugar chain obtained by the method according to any one of <1> to <4> on the same carrier by MALDI (matrix-assisted laser desorption/ionization) mass spectrometry.
<6> A method of mass spectrometry of a glycoprotein, comprising the steps of:

(1) obtaining the sugar chain excised by the method according to any one of <1> to <4>;

(2) fragmentating a peptide by dispensing a proteolytic enzyme solution on the solid-phased glycoprotein to obtain peptide fragments; and

(3) detecting the excised sugar chain obtained in (1) and the peptide fragments obtained in (2) on the same carrier by MALDI (matrix-assisted laser desorption/ionization) mass spectrometry.

According to the present invention, it is possible to provide a method of enzymatic reaction on a membrane by a sugar chain releasing enzyme, for an MALDI-TOF MS analysis of a glycoprotein that is solid-phased on a membrane is conducted directly on the membrane; a method of mass spectrometry of a sugar chain in which a sugar chain, for the MALDI-TOF MS analysis of a sugar chain excised from a glycoprotein that is solid-phased on a membrane is conducted directly on the membrane; and a method of mass spectrometry of a glycoprotein, for the MALDI-TOF MS analysis of a glycoprotein of a glycoprotein that is solid-phased on a membrane is conducted directly on the membrane. According to the present invention, it is possible to directly subject a glycoprotein that is solid-phased on a carrier to mass spectrometry, and omit the labors of extraction and purification of sugar chains, so that a mass spectrometric method of high throughput can be provided.

### BRIEF DESCRIPTION OF THE DRAWINGS

FIG. 1 is a printing image of a PNGase F solution and a trypsin solution onto protein spots.

FIG. 2 shows (a) a mass spectrum of a sugar chain obtained by printing of PNGase F with respect to ovalbumin, and (b) an identification result of N-glycan by GlycoMod Search.

FIG. 3 shows (a) a mass spectrum of a peptide fragment obtained by printing of trypsin with respect to ovalbumin, and (b) an identification result of a protein by Mascot Search.

FIG. 4 shows (a) a mass spectrum of a sugar chain obtained by printing of PNGase F with respect to transferrin, and (b) an identification result of N-glycan by GlycoMod Search.

FIG. 5 shows (a) a mass spectrum of a peptide fragment obtained by printing of trypsin with respect to transferrin, and (b) an identification result of a protein by Mascot Search.

FIG. 6 shows a mass spectrum of a sugar chain obtained by printing of PNGase F with respect to fetuin.

FIG. 7 shows (a) a mass spectrum of a sugar chain obtained by printing of PNGase F with respect to fetuin subjected to sialidase treatment, and (b) an identification result of N-glycan by GlycoMod Search.

### MODES FOR CARRYING OUT THE INVENTION

### Method of Excising Sugar Chain

In a method of excising a sugar chain according to the present invention, sugar chains of a glycoprotein that are solid-phased on a carrier is excised. The method of excising a sugar chain according to the present invention is particularly advantageously used when excised sugar chains are subjected to mass spectrometry directly on the membrane.

As the carrier, at least one selected from a membrane, a plate, nonmagnetic particles, magnetic particles and the like is used. When a membrane is used, a glycoprotein may be electrically transferred to the membrane for use. As the membrane, organic synthetic polymers such as polyvinylidene difluoride (PVDF), nitrocellulose, polyamide and polyethylene, and derivatives thereof may be recited. As polyamide, nylon and the like may be recited.

As the plate, a glass plate, a resin plate, a metal plate and the like may be recited. When such a plate is used, gel after electrophoresis of a sample containing a glycoprotein, or a membrane to which a glycoprotein is transferred may be used in contact with the surface of the plate.

When the excising method of a sugar chain according to the present invention is used for mass spectrometry, a metal plate, for example, a sample plate for mass spectrometry is preferably used as the carrier. Using a sample plate for mass spectrometry as the carrier is preferred in that mass spectrometry can be carried out on the same carrier after excision of a sugar chain. In such a case, a glycoprotein that is solid-phased on the sample plate for mass spectrometry may be obtained by bringing gel obtained by electrophoresis of a sample containing a glycoprotein into contact with a sample plate for mass spectrometry. It may be obtained by attachment, for example, by bonding a membrane to which a glycoprotein is transferred (from gel after electrophoresis or the like), to a sample plate for mass spectrometry. For attachment, bonding with a conductive dual-side adhesive tape may be used.

Further, as a carrier using non-magnetic particles, a carrier using polysaccharide gel, a synthetic polymer or the like is used. As a carrier using magnetic particles, those using an electric conductive magnetic metal as a base structure of the carrier serving as a carrier to be transferred after electrophoresis are used.

In the present invention, a preferred examples of a glycoprotein that is solid-phased on a carrier include those obtained by developing a sample containing a glycoprotein by electrophoresis, followed by transferring to a PVDF membrane, and attaching the transferred PVDF membrane to a sample plate for mass spectrometry.

Examples of the sugar chain releasing enzyme include, but are not limited to, peptide N-glycanase and glycopeptidase A and the like. The sugar chain releasing enzyme is used in the form of a sugar chain releasing enzyme solution contained in a reaction buffer solution satisfying the following conditions. A concentration of the sugar chain releasing enzyme in the sugar chain releasing enzyme solution is not particularly limited, and may be appropriately set by a person skilled in the art so that a desired enzyme amount is contained in a dispensing region. For example, when an enzyme amount of 5 microunits is to be contained in a dispensing region, the enzyme solution may be prepared so that the concentration is 100 milliunits/mL when the dispensing amount is 50 nanolitters.

The reaction buffer solution of the present invention substantially lacks the component that inhibits ionization of a sugar chain and the component that interrupts detection of a peak derived from a sugar chain on a mass spectrum when excised sugar chains are analyzed by MALDI mass spectrometry directly on the membrane.

As the component that inhibits ionization of a sugar chain, sodium phosphate, potassium phosphate and the like are recited. These salt components are generally often used as buffering agents, however, they remain on the membrane because of their non-volatility. Therefore, crystallization of a matrix on the membrane is inhibited, and ionization of a sugar chain is inhibited. Also a surfactant is the component that inhibits ionization of a sugar chain.

As the component that interrupts detection of a peak derived from a sugar chain on a mass spectrum, EDTA, a surfactant and the like may be recited. Such components as EDTA and a surfactant contained in the buffer solution will result in detection of a strong peak of EDTA or the surfactant in a mass spectrum, and a peak derived from a sugar chain is failed to be detected because it is blocked by noises. This inhibits detection of a peak derived from a sugar chain.

Therefore, the reaction buffer solution of the present invention substantially lacks the aforementioned components as a buffering agent. In other words, the buffering agent that is used in the reaction buffer solution of the present invention is substantially selected from volatile substances.

The expression “substantially lack the aforementioned components” means that the aforementioned components are allowed to be contained in such a degree that the components will not inhibit ionization of a sugar chain or interrupt detection of a peak derived from a sugar chain. For example, as the component that inhibits ionization of a sugar chain, such a very small amount as up to 25 picomoles is acceptable for the case of a buffering agent of a phosphate buffer solution, and up to 1 picomole for the case of a buffering agent of a Tris-HCl buffer solution in terms of a total amount contained in one dispensing region. As to the component that interrupts detection of a peak derived from a sugar chain, such a very small amount as up to 0.5 picomoles is acceptable for the case of EDTA or a surfactant, for example, in terms of a total amount contained in one dispensing region. Acceptable amounts of non-volatile components other than the above may be appropriately specified. Although the reaction buffer solution of the present invention permits containment of a certain degree of non-volatile components as described above, it is preferably a reaction buffer solution consisting exclusively of a volatile component and completely lacking any of such components as described above.

The reaction buffer solution of the present invention containing a buffering agent consisting essentially of a volatile substance has a buffer system such as a volatile weak acid and a volatile conjugate base, or a volatile weak base and a volatile conjugate acid, for example. The term “volatile” used herein refers to the property that the entity will completely vaporize within 30 minutes to a day at room temperature (20 to 37° C.) under reduced pressure of about 0.1 Pa (corresponding to reduced pressure by a standard rotary pump). On the other hand, the term “non-volatile” used herein refers to the property that the entity is in the form of solid or liquid under the above conditions.

The buffer solution of the present invention contains the following components. As a volatile weak acid component, one or more kind(s) is/are selected from formic acid, lactic acid, glycolic acid, benzoic acid, acetic acid, propionic acid, boric acid and the like; as a volatile weak base component, one or more kind(s) is/are selected from ammonia, methylamine, dimethylamine, trimethylamine, aniline, pyridine and the like; and as a volatile salt component, one or more kind(s) is/are selected from an ammonium ion, a pyridinium ion and the like, and a hydrogen carbonate ion, an acetate ion, a phenolate ion, a borate ion, a benzoate ion and the like.

These components are appropriately combined to establish an optimum pH for reaction of a sugar chain releasing enzyme. A Concrete pH may be from 7 to 9, and preferably from 8 to 9. The reaction buffer solution of the present invention is concretely selected from an (NH₄)HCO₃ buffer solution, a CH₃CO₂NH₄ buffer solution, an (NH₄)₂CO₃ buffer solution, a buffer solution using N(CH₃CH₂)₃ and the like. The buffer solutions recited above may be used singly or in combination of plural kinds.

A concentration of the reaction buffer solution used in the present invention is preferably about 10 to 50 mM, for example, about 25 mM.

For example, in the present invention, when a commercially available N-glycanase is used, the following operation is conducted. A commercially available N-glycanase (for example, PNGase F available from TaKaRa) contains EDTA and sodium phosphate. After removing these substances, for example, by ultrafiltration, and is used as a reaction buffer solution of the present invention, for example, as a sugar chain releasing enzyme solution replaced by 25 mM of ammonium bicarbonate.

For adding such a sugar chain releasing enzyme solution dropwise to a carrier, a microdispenser may be used. As the microdispenser, a device equipped with a piezo element or the like, as is used in an ink jet method may be used. As such a device, Chemical printer CHIP-1000 (available from SHIMADZU) and the like are recited.

When a microdispenser is used in the present invention, it is possible to control the liquid amount added by a single dispensing operation through one inkjet discharge part to about 100 pl, for example. Depending on the mechanism of inkjet, the amount may be controlled to be smaller. Also a desired liquid amount may be added dropwise by repeated discharge. For a specific one region, for example, a liquid amount of a nano litter level may be dispensed.

Further, by using a microdispenser, it is possible to make a extremely small dispensing range of about 7800 μm² by a jet of about 100 pl, for example. This dispensing range may be limited to a further smaller range by a mechanism of inkjet. Therefore, accurate dropping to the target position on the carrier can be realized. In the present invention, concretely, the dispensing range is preferably smaller than 0.5 mm in diameter. A dispensing range of less than 0.5 mm in diameter is as small as it occupies less than about 20% of an area of one protein spot.

Further, by changing the relative position on the carrier of the inkjet discharge part, the dispensing position can be shifted, so that it is possible to dispense a solution for different protein spots on the carrier.

Not being limited to the sugar chain releasing enzyme solution, any solutions that are added dropwise onto the carrier, such as other enzyme solutions, hydrophilizing agent solutions, water, matrix solutions as will be described later may be dispensed by using this microdispenser.

Concretely, a dispensing amount of the sugar chain releasing enzyme solution may be adjusted so that more than 5 microunits, preferably 25 microunits or more, for example, 25 microunits of the sugar chain releasing enzyme is contained in one dispensing region, although it depends on the amount of a glycoprotein subjected to development. With such an amount, a sufficient enzymatic reaction can be achieved, and an S/N ratio is favorably obtained from the integrated spectrum of 1000 shots in detected peaks in mass spectrometry, and the noise is insignificant. The upper limit of the dispensing amount of the sugar chain releasing enzyme solution is not particularly limited. For example, the above favorable result that is comparable with the case of an amount of the sugar chain releasing enzyme of 25 microunits is obtained with the amount of the sugar chain releasing enzyme of 50 microunits. In this manner, since no significant change is observed in the noise and the S/N ratio of mass spectrum even when the amount of the sugar chain releasing enzyme is increased, the dispensing amount may be appropriately determined by a person skilled in the art. Contrarily, the amount of the sugar chain releasing enzyme of 5 microunits or less results in high noise in the spectrum obtained in mass spectrometry.

As a hydrophilizing agent of the protein spot, n-octyl-β-D-glucopyranoside or polyvinylpyrrolidone may be used. Of these, polyvinylpyrrolidone is preferred because a sugar chain excision reaction occurs stably and reproducibility is high. As polyvinylpyrrolidone, those having a molecular weight, from polyvinylpyrrolidone 360 to polyvinylpyrrolidone 40 may be widely used, and any polyvinylpyrrolidone having the above range of molecular weight offers a similar favorable reaction effect.

The hydrophilizing agent may be used while it is dissolved in a buffer solution as described above or in a volatile organic solvent. When a volatile organic solvent is used, for example, methanol may be used, as an aqueous solution of about 60% (v/v), for example. The hydrophilizing agent may be used in a concentration of 0.1 to 5 (w/v) %, for example. In the case of polyvinylpyrrolidone, about 0.25 (w/v) % may be used. The use amount of the hydrophilizing agent solution may be appropriately determined by a person skilled in the art.

As conditions of sugar chain excision, usual conditions may be used. For example, the reaction may be caused at 20 to 37° C. for 30 minutes to overnight (e.g., 17 hours).

The glycoprotein that is solid-phased on a carrier according to the present invention may be preliminarily subjected to endoglycosidase treatment or exoglycosidase treatment prior to sugar chain excision by the sugar chain releasing enzyme. The endoglycosidase which is used in such a pretreatment is different from the aforementioned sugar chain releasing enzyme.

For example, as to a sugar chain having an acidic sugar chain, it is sometimes the case that conducting the endoglycosidase treatment or the exoglycosidase treatment is better. Some acidic sugar chains are difficult to be detected in positive mode mass spectrometry because of their negative charge. In addition, a large volume of a sample is sometimes required for detection of such acidic sugar chain even if mass spectrometry is conducted in a negative mode. In the case of such acidic sugar chain, it is preferred to conduct the endoglycosidase treatment or the exoglycosidase treatment.

Such treatment is conducted by dispensing a glycosidase solution containing endoglycosidase or exoglycosidase in a reaction buffer solution and hydrolyzing the acidic sugar. As a reaction buffer solution used for the glycosidase solution, a reaction buffer solution satisfying the same conditions as those used in excision of the sugar chain described above may be used at an optimum pH of glycosidase, or a usually used reaction buffer solution (i.e., a buffer solution containing EDTA or phosphate) may be used.

For example, when sugar chain fragments cut by the endoglycosidase treatment or the exoglycosidase treatment are later subjected to mass spectrometry on the same carrier, a reaction buffer solution that satisfies the same conditions as those used in the above sugar chain excision is used.

On the other hand, when sugar chain fragments cut by the endoglycosidase treatment or the exoglycosidase treatment are not later subjected to mass spectrometry on the same carrier, a normal buffer solution may be used. In such a case, following the glycosidase reaction, sugar chain fragments are removed by washing with water. In water washing, 5-minute water washing may be repeated about three times, for example.

Conditions for the endoglycosidase treatment or the exoglycosidase treatment may be those generally used in the art. For example, a reaction may be caused at 20 to 37° C. for 30 minutes to overnight (e.g., 17 hours).

One representative acidic sugar is sialic acid. Examples of a glycoprotein containing a sugar chain having sialic acid include fetuin, transferrin, an α1-acidic glycoprotein and the like.

For example, as N-glycan of fetuin, a double strand form and a triple strand form are known, and it is often the case that sialic acid binds to all ends of the sugar chain. Thus fetuin exhibits acidity and hence is difficult to be detected in a positive mode in mass spectrometry. Although detection in a negative mode is possible, it is sometimes necessary to prepare a large amount of a sample in order to increase the sensitivity. For this reason, in the case of fetuin, it is preferred to conduct sialidase treatment.

As to sialidase treatment, a very small dropping amount may be used because of high activity of sialidase. For example, a sialidase solution containing 0.25 milliunits of sialidase in a 0.1% (v/v) acetic acid aqueous solution may be used. As a result, sialic acid at ends of a sugar chain is hydrolyzed. The released sialic acid may be removed by washing with water because there is no need to detect the same. This treatment facilitates the subsequent mass spectrometry.

An α1 acidic glycoprotein may occasionally be detected without sialidase treatment because the sugar chain to which sialic acid binds is limited. Even if sialic acid binds, the number of sialic acid is one. For the sugar chain to which only one sialic acid binds, ionization in mass spectrometry is not so difficult even if sialidase treatment is not conducted. Therefore, for the case of the α1 acidic glycoprotein, sialidase treatment may or may not be conducted. The necessity thereof may be determined appropriately by a person skilled in the art.

Transferrin may also be detected without sialidase treatment. Therefore, as to transferrin, sialidase treatment may or may not be conducted. The necessity thereof may be determined appropriately by a person skilled in the art.

### <Method of Mass Spectrometry of Sugar Chain>

In a method of mass spectrometry of a sugar chain of the present invention, the aforementioned method of excising a sugar chain is used.

In brief, first, the aforementioned method is conducted to glycoprotein solid-phased on a carrier, to obtain excised sugar chain. Next, the excised sugar chains are subjected to MALDI mass spectrometry on the same carrier.

A matrix for use, a solvent for dissolving the matrix, a matrix concentration and the like may be appropriately determined by a person skilled in the art. For avoiding hydrolysis of sugar chains, it is preferred to use a solvent not containing TFA. As a concrete example, 2,5-DHB at a concentration of 10 mg/mL using a 25% acetonitrile aqueous solution may be recited. The matrix solution is added dropwise using a microdispenser as already described, to the same position as the position where the sugar chain releasing enzyme for excision is added dropwise. After drying the matrix solution, measurement is conducted by MALDI mass spectrometer to identify the sugar chains.

In the method of mass spectrometry of a sugar chain of the present invention, since the reaction buffer solution that essentially consists of a volatile substance as described above is used in the sugar chain excising step, ionization is not inhibited, and detection of a peak derived from the sugar chain is not interrupted. Therefore, it becomes possible to directly conduct MALDI mass spectrometry on the same carrier where sugar chain excision is conducted. In the obtained mass spectrum, no signal that is derived from a peptide is detected.

### <Method of Mass Spectrometry of Glycoprotein>

In the mass spectrometric method of a glycoprotein of the present invention, the aforementioned excising method of a sugar chain is used.

In brief, first, sugar chains are obtained that are excised from a glycoprotein solid-phased on a carrier by the aforementioned method.

Meanwhile, a proteolytic enzyme solution is added dropwise to a glycoprotein solid-phased on a carrier to obtain peptide fragments are obtained. At this time, a protein digestion enzyme is dispensed to the position located within the same spot as the protein spot used as an object for mass spectrometry of sugar chains, but is different from the position where the sugar chain releasing enzyme solution is dispensed for sugar chain excision. Conditions for digestion are not particularly limited, and may be appropriately determined by a person skilled in the art.

The matrix solution as already described is added dropwise to the position where sugar chain excision is conducted, and to the position where protein digestion is conducted, and then dried. The excised sugar chains are subjected to measurement by a MALDI mass spectrometer to identify the sugar chains. Also the peptides are subjected to measurement by a MALDI mass spectrometer to identify the peptides by a PMF analysis.

In the mass spectrometric method of a glycoprotein of the present invention, since the reaction buffer solution that essentially consists of a volatile substance as described above is used in the sugar chain excising step, in mass spectrometry of sugar chains, ionization is not inhibited, and detection of a peak derived from a sugar chain is not interrupted. Therefore, it becomes possible to directly conduct MALDI mass spectrometry on the same carrier where sugar chain excision is conducted. In the obtained mass spectrum of the sugar chain, no signal that is derived from a peptide is detected.

When mass spectrometry is conducted in the present invention, MS and multi-stage MS of MS/MS or more may be conducted. When multi-stage MS is conducted, it may be conducted by adjusting an amount of the sample or the like appropriately by a person skilled in the art.

### EXAMPLES

In the following, the present invention will be described more specifically by way of Examples, but the present invention will not be limited by these Examples.

### Example 1

Each 40 picomoles of four kinds of standard glycoproteins (ovalbumin, asialofetuin, transferrin, α1-acidic glycoprotein) were developed on a gel by one-dimensional electrophoresis (SDS-PAGE). Each separated glycoprotein was transferred electrically to a PVDF carrier from the gel. After dying with Direct Blue 71, the membrane was directly bonded to an MALDI-TOF target plate with the use of a double-sided conductive tape. In this manner, each glycoprotein that is solid-phased on the membrane was obtained.

PNGase F (available from TaKaRa) was ultra-filtrated, and a buffer solution was replaced by a 25 mM ammonium bicarbonate aqueous solution, to obtain a PNGase F solution. Separately, a trypsin solution was prepared. The PNGase F solution and the trypsin solution were printed (dispensed) to each glycoprotein spot that was solid-phased on the membrane with the use of CHIP-1000 (available from SHIMADZU).

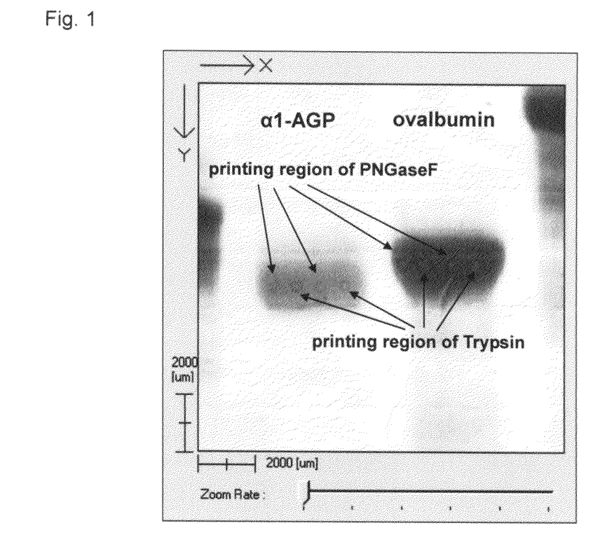

FIG. 1 shows spot parts of an α1-acidic glycoprotein (α1-AGP) and ovalbumin in an enlarged manner in the membrane after printing. By printing so that the PNGase F solution and the trypsin solution will not overlap with each other, two printing regions of the PNGase F solution and two printing regions of the trypsin solution were obtained per one protein spot as shown in FIG. 1. Every printing region was controlled to have a diameter of 0.5 mm or less.

A matrix solution (10 mg/mL, 2.5-DHB) was printed so that it overlaps with each printing position (sugar chain excision position and peptide digestion position). After drying, analysis was conducted directly on the membrane with the use of a MALDI-QIT-TOF mass spectrometer AXIMA-QIT (available from SHIMADZU).

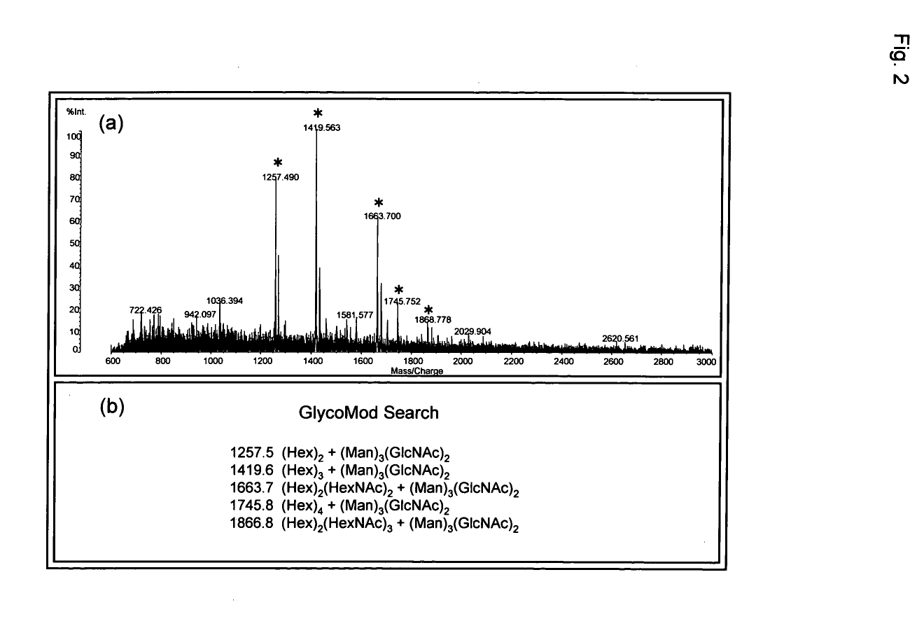

Of the mass spectrums obtained in the above Example, FIG. 2(a) shows a mass spectrum of the sugar chain obtained by printing of PNGase F with respect to ovalbumin.

### Identification Results by GlycoMod Search

(http://au.expasy.org/tools/glycomod/) for peaks marked with * in the mass spectrum of FIG. 2(a) ((m/z)=1257.5, 1419.6, 1663.7, 1745.8, 1866.8) are shown in FIG. 2(b).

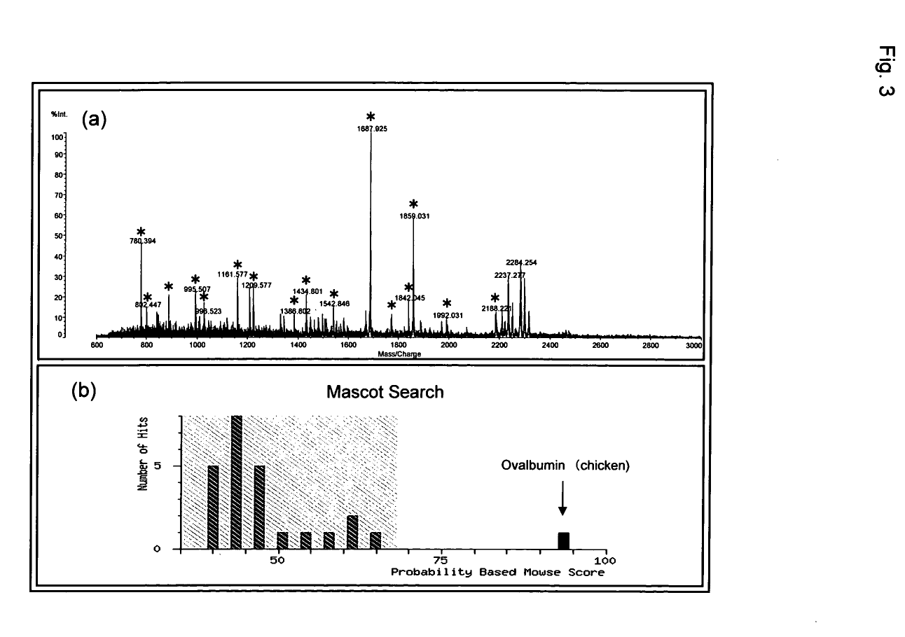

A mass spectrum of the peptide fragment obtained by printing of trypsin with respect to ovalbumin is shown in FIG. 3(a). FIG. 3(b) shows identification results of a protein by Mascot Search (http://www.matrixscience.com/search_form_select.html) for the peaks marked with * in the mass spectrum of FIG. 3(a). As shown in FIG. 3(b), ovalbumin (Ovalbumin (chicken): arrow) was hit at the highest score.

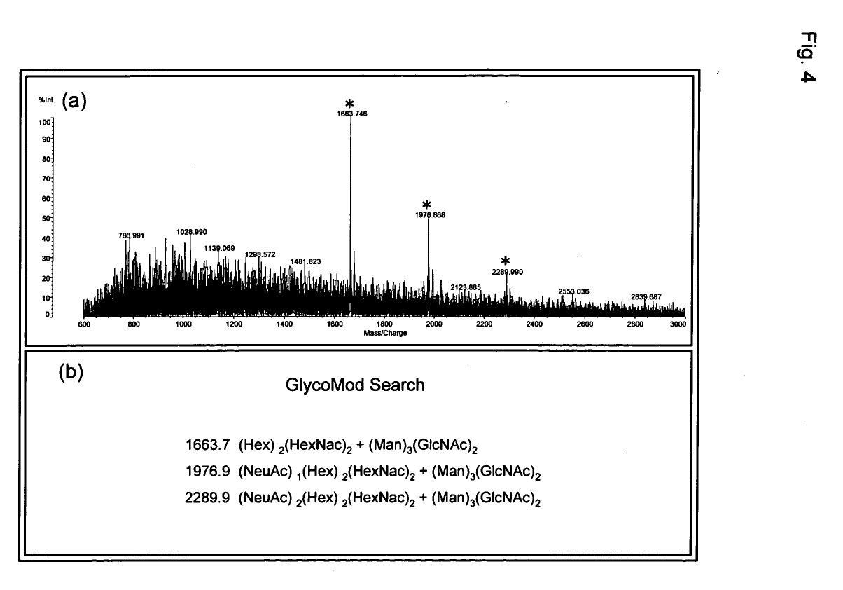

FIG. 4(a) shows a mass spectrum of the sugar chain obtained by printing of PNGase F with respect to transferrin. FIG. 4(b) shows identification results for peaks marked with * in the mass spectrum of FIG. 4(a) ((m/z)=1663.7, 1976.9, 2289.9) by GlycoMod Search.

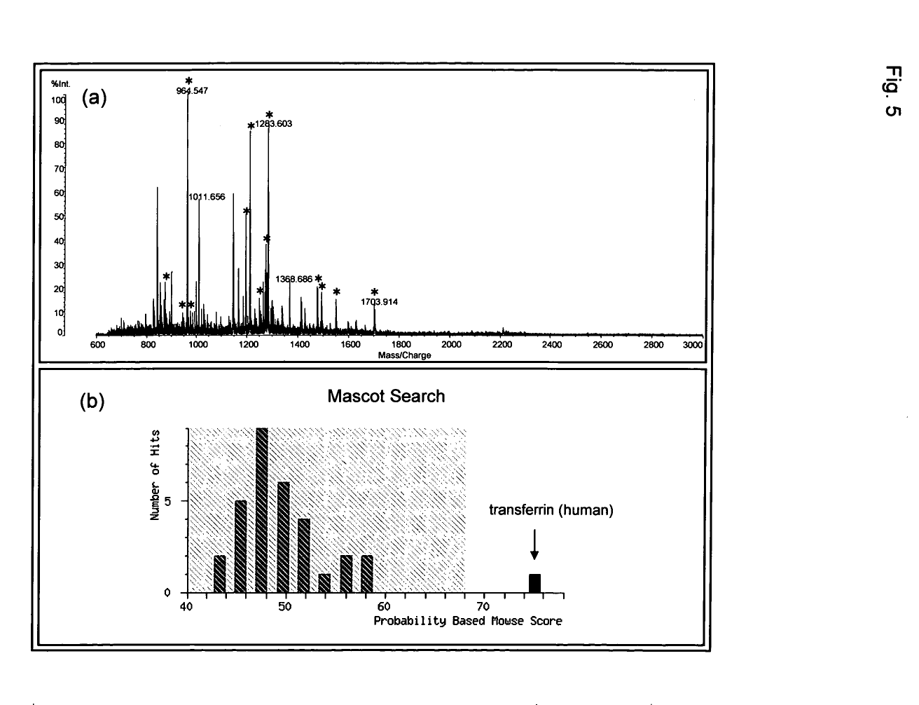

FIG. 5(a) shows a mass spectrum of the peptide fragment obtained by printing of trypsin with respect to transferrin. FIG. 5(b) shows identification results of a protein by Mascot Search of FIG. 5(a). As shown in FIG. 5(b), transferrin (transferrin (human): arrow) was hit at the highest score.

In this way, for every glycoprotein, N-glycan as already reported was detected in the sugar part, and the protein part was identified at a high score.

### Example 2

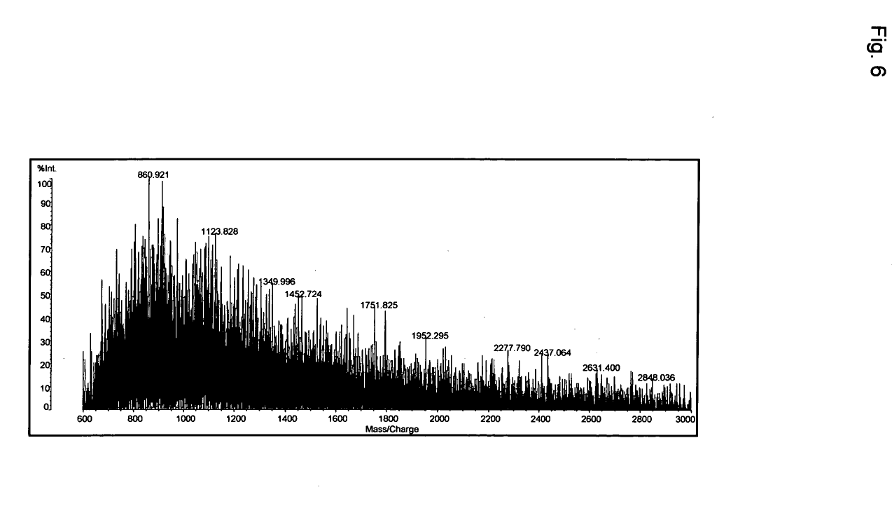

First, using 40 picomoles of fetuin having sialic acid, mass spectrometry for a sugar chain was conducted in the same manner as in Example 1. The obtained mass spectrum is shown in FIG. 6. As shown in FIG. 6, only a background noise was observed and no signal for a sugar chain was obtained.

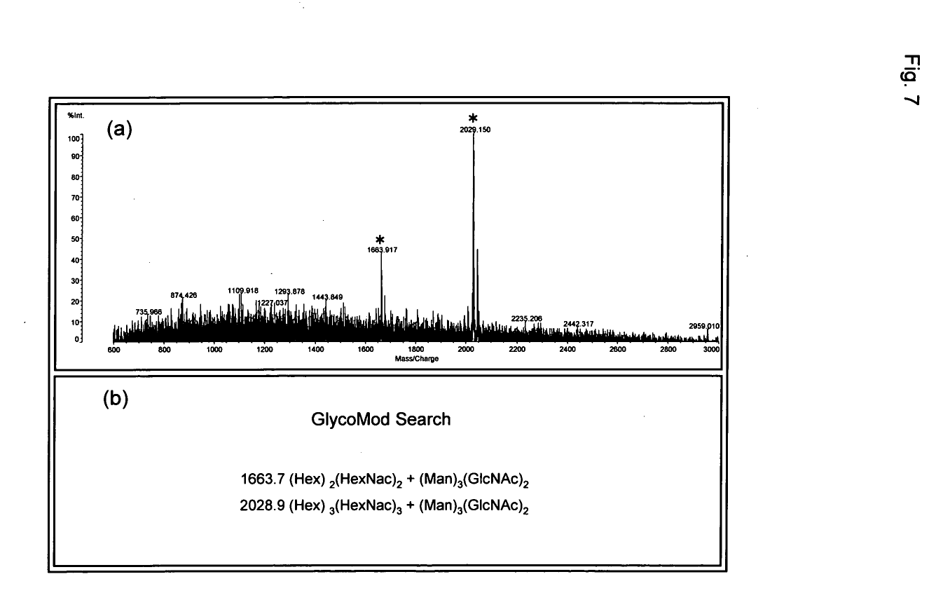

Next, the mass spectrometry of sugar chain was conducted in the same manner as Example 1, except that 40 picomoles of fetuin having sialic acid was used, sialidase (neuraminidase, available from Nacalai Tesque) (0.25 milliunit quantity, 0.1% acetic acid aqueous solution) was printed to conduct a reaction of cutting sialic acid, the membrane was washed with pure water (5 minutes×3 times), followed by drying, and then PNGase F treatment was conducted. The obtained mass spectrum is shown in FIG. 7(a). FIG. 7(b) shows identification results of peaks marked with * in mass spectrum of FIG. 7(a) ((m/z)=1663.7, 2028.9) by GlycoMod Search. As shown in FIG. 7(b), a signal derived from a sugar chain can be obtained by cutting the sialic acid.

In the above Examples, ovalbumin, asialofetuin, transferrin, an α1-acidic glycoprotein, and fetuin were selected as objective glycoproteins to be analyzed, and N-glycanase was used as a sugar chain releasing enzyme while an ammonium bicarbonate aqueous solution was used as a reaction buffer solution. The present invention, however, may be applied to a glycoprotein other than the above, a sugar chain releasing enzyme other than N-glycanase, and a reaction buffer solution other than the ammonium bicarbonate aqueous solution. Therefore, these Examples are merely illustrative in all respects, and should not be interpreted as limitative. Further, all modifications made within the range of equivalents of claims are embraced in the present invention.

1. A method of excising a sugar chain to obtain the excised sugar chain by excising the sugar chain by dispensing a sugar chain releasing enzyme solution on a glycoprotein that is solid-phased on a carrier,
wherein the sugar chain releasing enzyme solution is a solution containing a sugar chain releasing enzyme in a reaction buffer solution containing a buffering agent consisting essentially of a volatile component.

2. The method of excising a sugar chain according to claim 1, wherein the sugar chain releasing enzyme is N-glycanase or glycopeptidase A.

3. The method of excising according to claim 1, wherein the reaction buffer solution is selected from an (NH₄)HCO₃ buffer solution, a CH₃CO₂NH₄ buffer solution, an (NH₄)₂CO₃ buffer solution, and a N(CH₃CH₂)₃ buffer solution.

4. The method of excising a sugar chain according to claim 1, wherein the solid-phased glycoprotein is treated with endoglycosidase or exoglycosidase other than the sugar chain releasing enzyme.

5. A method of mass spectrometry of a sugar chain which detects the excised sugar chain obtained by the method according to any one of claims 1 to 4 on the same carrier by MALDI (matrix-assisted laser desorption/ionization) mass spectrometry.

6. A method of mass spectrometry of a glycoprotein, comprising the steps of:
(1) obtaining the sugar chain excised by the method according to any one of claims 1 to 4;
(2) fragmentating a peptide by dispensing a proteolytic enzyme solution on the solid-phased glycoprotein to obtain peptide fragments; and
(3) detecting the excised sugar chain obtained in (1) and the peptide fragments obtained in (2) on the same carrier by MALDI (matrix-assisted laser desorption/ionization) mass spectrometry.
